(19) United States
(12) Patent Application Publication
(10) Pub. No.: US 2022/0011285 A1
(43) Pub. Date: Jan. 13, 2022

(54) METHOD TO DETERMINING METAL CATIONS IN WATER

(71) Applicant: Kemira Oyj, Helsinki (FI)

(72) Inventors: Salla PUUPPONEN, Espoo (FI); Sari KRAPU, Espoo (FI)

(21) Appl. No.: 17/281,842

(22) PCT Filed: Sep. 27, 2019
(86) PCT No.: PCT/FI2019/050690

§ 371 Date: Mar. 31, 2021

(30) Foreign Application Priority Data
Oct. 1, 2018 (FI) 20185818

(51) Int. Cl. G01N 33/18; G01N 1/34

(52) U.S. Cl. CPC G01N 33/1813; G01N 1/34

(57) ABSTRACT

The present invention relates to utilization of lanthanide time resolved fluorescence for determining concentration of trivalent metal ions in a sample. In the method sample comprising trivalent metal ions is admixed with a reagent comprising a lanthanide (lll) ion and a chelating agent. The trivalent metal ion in the sample is allowed to interact with the reagent comprising the lanthanide (lll) ion and the chelating agent, followed by exciting the sample and detecting a signal deriving from the lanthanide (lll) ion, and determining the concentration of the trivalent metal ion in the sample by using the detected signal.

### FIELD OF THE INVENTION

The present invention relates to utilization of lanthanide time resolved fluorescence for determining concentration of metal ions having an absolute oxidation state+III or higher in samples.

### BACKGROUND

Metals in waste water streams can pollute rivers and lakes. Many metals are potentially poisonous to aquatic life or may slow their development or even result in death. Metal contamination and its treatment are thus critical in waste water treatment. Particularly toxic heavy metals, such as chromium, cobalt and manganese must be often controlled and measured. In addition to analyses of toxic and harmful metals, quantification of metal ions causing corrosion or scaling problems is often needed to be analyzed in order to e.g. optimize the scale or corrosion treatment of many processes.

Quantification of metal ion containing products, such as aluminum or iron based coagulants, is also important in order to optimize the dosaging of the product or to detect the residual product present in the waters after use.

Metals are typically analyzed using conventional analysis technologies, such as atomic absorption spectrometry (AAS), inductively coupled mass spectrometry or atomic emission spectrometry (ICP-MS, ICP-AES). The conventional analysis methods are laborious, the equipment is expensive and they require typically experienced personnel to conduct the measurements.

There are also several spectrophotometric or fluorescence methods for metal quantification. These methods are often quick and robust, but they may also suffer from interference from the measurement matrix or long pretreatment procedures.

Based on above there is need for quick and simple method determining trivalent metal ions in a sample.

### SUMMARY OF INVENTION

An object of the present invention is to provide a method determining metal ions having an absolute oxidation state+III or higher in samples.

Another object of the present invention is to provide a quick and simple method determining metal ions having an absolute oxidation state+III or higher in samples.

The present invention provides a quantification method for specific metals based on time resolved fluorescence (TRF) of lanthanide chelates. The use of TRF removes typical short-lived, interfering fluorescence signal possibly present in the sample medium by temporal resolution (the fluorescence signal is not recorded immediately but after a waiting period or lag time). Lanthanide ions do not only have exceptionally long fluorescence lifetime, but they also have narrow banded emission lines and long Stokes' shift. Alone, lanthanide ions have very low energy absorption. The absorptivity of the lanthanides can be substantially increased by chelating the trivalent lanthanide ion with energy mediating ligands. In aqueous solutions, the ligands increase the absorptivity and protect the lanthanide ion from water molecules that quench the fluorescence signal by radiationless decay process of lanthanide and OH groups of water

The inventors surprisingly found that TRF of lanthanide chelates can be utilized for metal quantification. It was surprisingly found that the trivalent metal ions quench the TRF signal of lanthanide chelates efficiently due to the similar charge of the lanthanide and the metal to be analyzed. The metals substitute the chelation sites of the chelation agents, decreasing the amount of lanthanide ions chelated to the ligand resulting in lower TRF signal of lanthanide chelates.

The signal of unknown sample containing unknown amount of metal ions to be analyzed is compared to signal of known sample comprising known amount of metal ions. Preferably, the metal ions are trivalent, such as aluminum(III), iron(III) or chromium(III) ions. In addition, metals that are or can be oxidized to oxidation state III or higher can also be measured. Examples of metal ions having oxidation state higher than III are aluminum and chromium.

### BRIEF DESCRIPTION OF FIGURES

FIG. 1 illustrates schematic presentation of the TRF measurement principle in the method of the present invention.

FIG. 2 illustrates TRF signal of SASMAC chelated europium as a function of added aluminum and iron.

### DETAILED DESCRIPTION

The present invention provides a method determining metal ions in a sample. More particularly the present invention provides method for determining concentration of metal ions having an absolute oxidation state+III or higher in a sample comprising metal ions having absolute oxidation state+III or higher, the method comprising
optionally diluting and/or purifying the sample,
admixing the sample with a reagent comprising lanthanide(III) ion and chelating agent or chelating agents,
allowing the metal ion in the sample to interact with the reagent comprising the lanthanide(III) ion and the chelating agent or chelating agents
exciting the sample at a excitation wavelength and detecting a sample signal deriving from the lanthanide(III) ion at a signal wavelength by using time-resolved fluorescence determining the concentration of the metal ions in the sample by using the detected sample signal.

Preferably the metal ions are metal ions having an absolute oxidation state+III.

In one embodiment the reagent comprising lanthanide(III) ion and the chelating agent or chelating agents are admixed together prior admixing with the sample.

In other embodiment the sample and the chelating agent or chelating agents are admixed together prior admixing with the reagent comprising lanthanide(III) ion.

In other embodiment the reagent comprising lanthanide(III) ion and the sample are admixed together prior admixing with the chelating agent or chelating agents.

The metal ion is selected from a group consisting of titanium, chromium, manganese, iron, cobalt, nickel, gold and aluminum, preferably a trivalent metal ion selected from aluminum, iron and chromium.

In one embodiment concentration of the metal ion in the measurement mixture is in the range of 0.005-50 ppm, preferably 0.1-15 ppm, and more preferably 0.5-5 ppm.

In case the concentration of the metal ion in the sample is higher, the sample can be diluted.

The lanthanide(III) ion is selected from europium, terbium, samarium or dysprosium ions, preferably europium or terbium ions.

In a preferred embodiment the reagent comprises a lanthanide(III) salt. The lanthanide(III) salt is selected from halogenides and oxyanions, such as nitrates, sulfates or carbonates, preferably from hydrated halogenides or nitrates, more preferably chloride.

In one embodiment concentration of the lanthanide(III) ion in the measurement mixture is in the range 0.1-100 μM, preferably 0.1-50 μM, and more preferably 1-20 μM.

the chelating agent comprises at least one or more functional groups capable of chelating the lanthanide(III) and the metal ions, preferably one or more groups selected from esters, ethers, thiols, hydroxyls, carboxylates, sulfonates, amides, phosphates, phosphonates, amines or any combinations thereof.

In an embodiment, chelating agent contains additionally aromatic group or groups. The aromatic group(s) amplifies the signal of the lanthanide(III) ion.

Examples of suitable chelating agents are ethyleneamines, such as diethyleneamine and triethylenetetramine, EDTA, DPA, deferoxamine, deferiprone, phthalate, salicylate complexes such as dipicolinic acid, polyacrylic acid/polyacrylate/polymaleate and copolymers of these.

In one embodiment concentration of the chelating agent in the measurement mixture is in the range of 0.01-500 ppm, preferably 0.1-500 ppm, and more preferably 1-200 ppm.

By term “measurement mixture” is meant the admixture in the measurement.

In one embodiment pH value of the sample is adjusted to a level in range between pH 3 and pH 8, preferably in range from pH 5 to pH 7.5.

In a preferred embodiment buffer is used in the measurement for standardization of the pH. The buffering agent is selected from a group consisting of Good's zwitterionic buffering agents, bis-trispropane, piperazine-N,N′-bis(2-ethanesulfonic acid) (PIPES), cholamine chloride, 2-morpholinopropanesulfonic acid (MOPS), 2-hydroyxy-3-morpholin-4-ylpropane-1-sulfonic acid (MOPSO), 4-(2-hydroxyethyl)-1-piperazineethanesulfonic acid (HEPES), glycinamide, glycylglycine, bicine and 3-(cyclohexylamino)-1-propanesulfonic acid (CAPS), preferably HEPES. The pH should not be excessively alkaline in order to prevent possible precipitation of the lanthanide hydroxides.

The sample is optionally diluted to suitable aqueous solution e.g. deionized water or brine containing monovalent and/or divalent ions. Preferably, the dissolution brine does not contain any trivalent ions. Preferably the sample is an aqueous solution.

The sample is optionally purified by using a purification method selected from centrifugation, size exclusion chromatography, cleaning with solid-phase extraction (SPE) cartridges, dialysis techniques, extraction methods for removing hydrocarbons, filtration, microfiltration, ultrafiltration, nanofiltration, membrane centrifugation, pH adjustment, reductive/oxidative pretreatment, removal of interfering compounds by chelation/complexation or precipitation, and any combinations thereof.

If the sample comprises metal ions having absolute oxidation state+II said metal ions can optionally be oxidized to absolute oxidation state+III prior admixing the sample with the reagent comprising lanthanide(III) ion and the chelating agent.

If the sample comprises metal ions having absolute oxidation state higher than +III said metal ions can optionally be reduced to absolute oxidation state+III prior admixing the sample with the reagent comprising lanthanide(III) ion and the chelating agent.

Unknown concentration of the metal ion in the sample is determined by comparing the sample signal to calibration curve. The calibration curve is obtained from TRF measurement of calibration standard samples with varying metal ion concentrations. Same dilution and or purification steps and measurement parameters have to be used for both the sample and calibration samples.

The lanthanide(III) ion is excited at excitation wavelength and measured at emission wavelength and detected by using time-resolved fluorescence (TRF). Any TRF reader can be employed. Excitation and emission wavelengths are selected so that the S/N is the best. Suitable delay time can be optimized.

The excitation and emission wavelengths and the delay time are chosen based on the requirements of the lanthanide ion.

In an exemplary embodiment excitation wavelength and emission wavelength and delay time for Europium is 395 nm and 615 nm and 400 μs respectively.

The present invention further provides use of the method of the present invention for determining concentration of metal ions having an absolute oxidation state+III or higher in a sample.

The sample can originate from geothermal processes, cooling towers, desalination plants, water treatment processes, mining or agricultural industry, pulp/paper mill effluent, waste streams of food industry and oil and gas production.

The present invention further provides a device comprising means for performing the method of the present invention for determining concentration of metal ions having an absolute oxidation state+III or higher in a sample

The examples are not intended to limit the scope of the invention but to present embodiments of the present invention.

### Examples

### Example 1. Measurement of TRF Signal of SASMAC Chelated Europium as a Function of Added Aluminum and Iron in Known Amounts

Salt containing trivalent cation FeCl₃ or AlCl₃ is used is diluted into suitable concentration range. The chelating agent sodium allyl sulphonate maleic acid anhydride (SASMAC) is diluted into suitable concentration.

EuCl₃ and HEPES were used as lanthanide source and buffer, respectively. The pH of the buffer solution was modified to pH 7.4.

Concentrations of SASMAC polymer, Eu and HEPES were 49 ppm, 0.011 mM and 5 mM, respectively, in the measurement. The concentration of the trivalent cation was varied between 2.5 and 4500 μM in the measurement.

The reagents were diluted into brine. Brine composition used in this example is presented in Table 1.

The trivalent salt solution and chelate may be first mixed together, after which the lanthanide and buffer are added, or the chelate may be first admixed with lanthanide prior to addition of the analyte solution comprising the trivalent cations. The trivalent metal ion in the sample is allowed to interact with the reagent comprising the lanthanide(III) ion and the chelating agent, followed by exciting the sample at a excitation wave length 395 nm and detecting a sample signal deriving from the lanthanide(III) ion at a signal wave length 615 nm by using time-resolved fluorescence (delay time for europium was 400 μs) and determining the concentration of the metal ions in the sample by using the detected sample signal.

In FIG. 2 is presented TRF signals of the Example 1.

The results obtained from Example 1 and presented as curves in FIG. 2 can be utilized when concentrations of iron or aluminum of unknown samples are determined by comparing signal of the unknown sample with the signal of known samples (calibration curve).

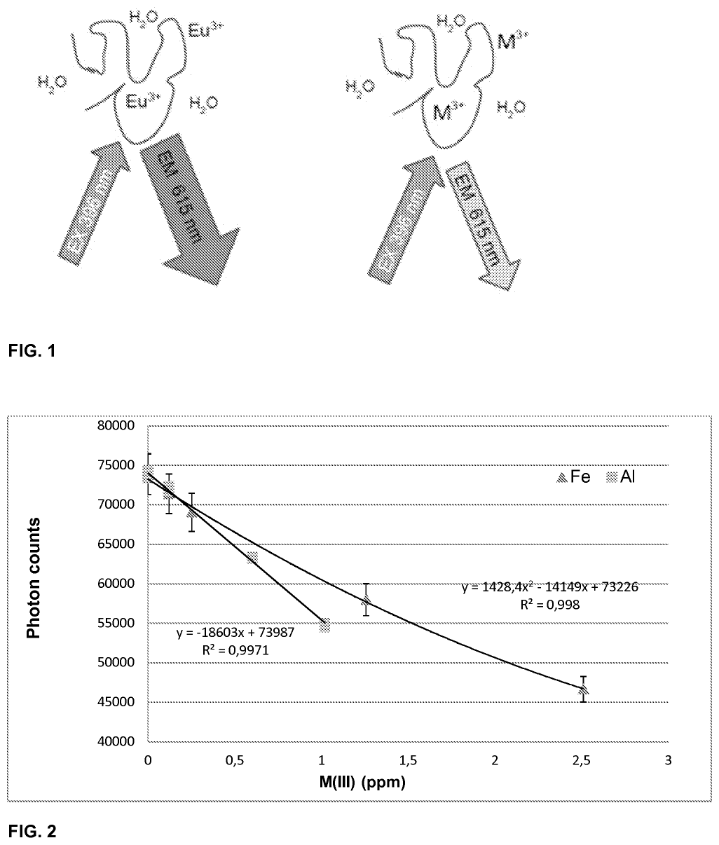

FIG. 1 illustrates schematic presentation of the TRF signal decrease due to trivalent metal ions.

TABLE 1
Brine composition used in Example 1

| Salt | Concentration (ppm) |
|---|---|
| NaCl | 35030.00 |
| CaCl₂*2H₂O | 2240.00 |
| MgCl2*6H₂O | 1460.00 |
| KCl | 210.00 |
| BaCl₂*2H₂O | 130.00 |

### Example 2. Measurement of Total Fe(II) and Fe(III) from Same Sample

Concentrations of Fe(II) and Fe(III) in the sample are unknown.

Step A: First, trivalent Fe(III) is measured similarly as in Example 1. No oxidative or reductive pretreatment methods are used.

Step B: After the measurement of Fe(III), the Fe(II) in the sample is oxidized to oxidation state+III. After the oxidation, the total iron is measured using the same methodology as in the Example 1. The quantity of Fe(II) is obtained by subtracting the concentration of Fe(III) (Step A) from the total iron signal measured in the Step B.

The total concentration of Fe(II) and Fe(III) is determined by comparing the sample signal with calibration curve.

Any metal ions that are commonly in oxidation states+II, such as chromium and cobalt, are first oxidized to oxidation state+III or higher and then analyzed using the same methodology as in previous examples.

### Example 3 Measurement of Total Fe and Al from the Same Sample

Step A: If sample contains Fe(II), it is oxidized to state+III in pretreatment. The sample matrix should not contain other cations that oxidize to oxidation state+III in the pretreatment. After the optional pretreatment, the Al and Fe are measured using the same methodology as in the Example 1. However, the ligand used is chosen so that the quench of the TRF signal is similar for both Al and Fe species.

Step B: After the measurement of Fe and Al, the Fe(III) in the sample is reduced to oxidation state+II. After reduction, the non-reduced Al(III) is measured using the same methodology as in the Example 1. The quantity of Fe is obtained by subtracting the concentration of Al+Fe (Step A) from the Al signal measured in the Step B.

The total concentration of Fe and Al is determined by comparing the sample signals with calibration curves.

### Example 4 Quantification of Oxidizable Metal Ions

Metals that can be oxidized into oxidation state+III or higher and form water soluble complexes in the oxidized state are first oxidized and then analyzed using the same methodology as in previous examples. The used ligand is chosen so that it forms water-soluble complexes preferably with the analyte

1. A method for determining concentration of metal ions having an absolute oxidation state+III or higher in a sample comprising metal ions having absolute oxidation state+III or higher, the method comprising:
optionally diluting and/or purifying the sample;
admixing the sample with a reagent comprising lanthanide(lll) ion and chelating agent or chelating agents;
allowing the metal ion in the sample to interact with the reagent comprising the lanthanide(lll) ion and the chelating agent or chelating agents; and
exciting the sample at a excitation wavelength and detecting a sample signal deriving from the lanthanide(lll) ion at a signal wavelength by using time-resolved fluorescence determining the concentration of the metal ions in the sample by using the detected sample signal.

2. The method according to claim 1, wherein the reagent comprising lanthanide(lll) ion and the chelating agent are or chelating agents admixed together prior admixing with the sample; or the sample and the chelating agent or chelating agents are admixed together prior admixing with the reagent comprising lanthanide(lll) ion; or the reagent comprising lanthanide(lll) ion and the sample are admixed together prior admixing with the chelating agent or chelating agents.

3. The method according to claim 1, wherein the metal ion is selected from the group consisting of titanium, chromium, manganese, iron, cobalt, nickel, gold and aluminum, preferably a trivalent metal ion selected from aluminum, iron and chromium.

4. The method according to claim 1, wherein the sample is pretreated by oxidizing metal ions having absolute oxidation state+II to absolute oxidation state+III or reducing metal ions having absolute oxidation state higher than +III to absolute oxidation state+III prior admixing the sample with the reagent comprising lanthanide(lll) ion and the chelating agent.

5. The method according to claim 1, wherein concentration of the lanthanide(lll) ion in the measurement mixture is in a range 0.1-100 μM, preferably 0.1-50 μM, and more preferably 1-20 μM.

6. The method according to claim 1, wherein concentration of the metal ion in the measurement mixture is in a range of 0.005-50 ppm, preferably 0.1-15 ppm, and more preferably 0.5-5 ppm.

7. The method according to claim 1, wherein concentration of the chelating agent in the measurement mixture is in a range of 0.01-500 ppm, preferably 0.1-500 ppm, and more preferably 1-200 ppm.

8. The method according to claim 1, wherein the chelating agent comprises at least one or more functional groups capable of chelating the lanthanide(III) and the metal ions, preferably one or more groups selected from esters, ethers, thiols, hydroxyls, carboxylates, sulfonates, amides, phosphates, phosphonates, amines or any combinations thereof; preferably the chelating agent contains additionally aromatic group or groups.

9. The method according to claim 1, wherein the lanthanide(lll) ion is selected from europium, terbium, samarium or dysprosium ions, preferably europium or terbium ions.

10. The method according to claim 1, wherein the reagent comprises a lanthanide(lll) salt, preferably halogenide or oxyanion, more preferably hydrated halogenides or nitrates, most preferably chloride.

11. The method according to claim 1, wherein the sample is purified by using a purification method selected from the group consisting of centrifugation, size exclusion chromatography, cleaning with solid-phase extraction (SPE) cartridges, dialysis techniques, extraction methods for removing hydrocarbons, filtration, microfiltration, ultrafiltration, nanofiltration, membrane centrifugation and any combinations thereof.

12. The method according to claim 1, wherein a pH value of the sample is adjusted to a level in a range between pH 3 and pH 8, preferably in a range from pH 5 to pH 7.5.

13. Use of the method according to claim 1 for determining concentration of metal ions having an absolute oxidation state+III or higher in a sample.

14. The use according to claim 13, wherein the sample originates from geothermal processes, cooling towers, desalination plants, water treatment processes, mining or agricultural industry, pulp/paper mill effluent, waste streams of food industry and oil and gas production.

15. A device comprising means for performing the method according claim 1 for determining concentration of metal ions having an absolute oxidation state+III or higher in a sample.
